(19) United States
(12) Patent Application Publication
(10) Pub. No.: US 2019/0029952 A1
(43) Pub. Date: Jan. 31, 2019

(54) OPHTHALMIC LENS AND METHOD FOR MANUFACTURING THE SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/783,055

(22) Filed: Oct. 13, 2017

(30) Foreign Application Priority Data
Jul. 27, 2017 (CN) 201710624664.9

(51) Int. Cl. A61K 9/00; A61K 9/70; A61K 47/34; A61K 47/32; A61K 31/498

(52) U.S. Cl. CPC A61K 9/0051; A61K 9/7007; A61K 31/498; A61K 47/32; A61K 47/34

(57) ABSTRACT

For the manufacture of an ophthalmic lens, the ophthalmic lens includes a gel matrix, at least one first polyelectrolyte coating layer, at least one second polyelectrolyte coating layer, and drugs. The first polyelectrolyte coating layer is formed on the gel matrix. The second polyelectrolyte coating layer is formed on the first polyelectrolyte coating layer. The drugs are in place between the first polyelectrolyte coating layer and the second polyelectrolyte coating layer. The first polyelectrolyte coating layer and the second polyelectrolyte coating layer interact electrostatically. When intraocular pressure (TOP) rises, the electrostatic interaction between the first polyelectrolyte coating layer and the second polyelectrolyte coating layer is broken and the drugs are released.

### FIELD

The subject matter generally relates to an ophthalmic lens and a method for manufacturing the ophthalmic lens.

### BACKGROUND

For cosmetic and portable purposes, many contact lenses have matrixes and one or more colored films printed on the matrixes. However, such contact lenses cannot timely detect intraocular pressure (IOP) levels for achieving a real-time drug release.

### BRIEF DESCRIPTION OF THE DRAWINGS

Implementations of the present technology will now be described, by way of example only, with reference to the attached figures.

FIG. 1 is a cross-sectional view of an exemplary embodiment of an ophthalmic lens of the present disclosure.

FIG. 2 is a flowchart of an exemplary embodiment of a method for manufacturing an ophthalmic lens.

FIG. 3 is a cross-sectional view of a matrix of the present disclosure.

FIG. 4 is a cross-sectional view of the matrix of FIG. 3, and a first polyelectrolyte solution and a first polyelectrolyte coating layer of the present disclosure.

FIG. 5 is a cross-sectional view of the matrix with the first polyelectrolyte coating layer of FIG. 4, a second polyelectrolyte solution, and a second polyelectrolyte coating layer of the present disclosure.

### DETAILED DESCRIPTION OF EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

The term “comprising,” when utilized, means “including, but not necessarily limited to”; it specifically indicates open-ended inclusion or membership in the so-described combination, group, series, and the like.

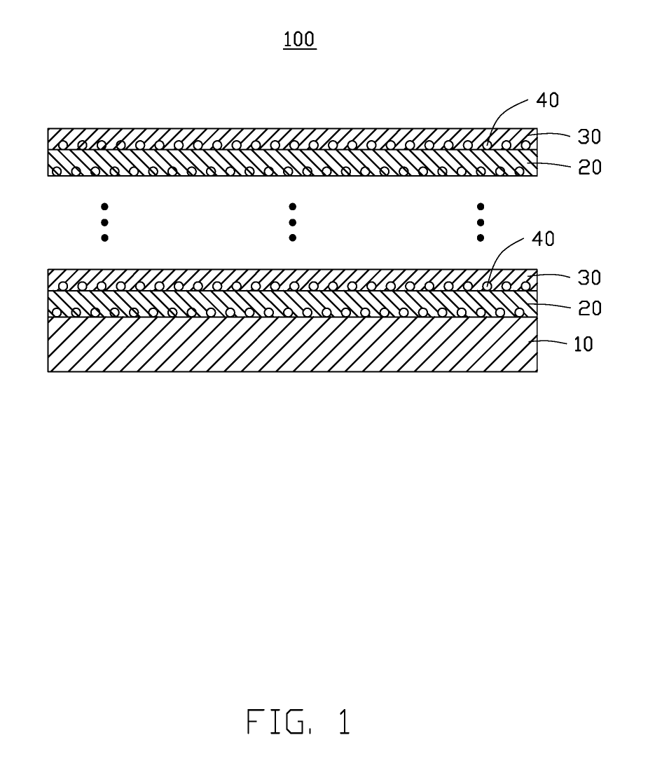

FIG. 1 illustrates an exemplary embodiment of an ophthalmic lens 100. The ophthalmic lens 100 may be rigid gas permeable (RGP) contact lens, soft gas permeable contact lens, ortho-K lens, or intraocular lens (IOL).

The ophthalmic lens 100 includes a gel matrix 10, at least one first polyelectrolyte coating layer 20, at least one second polyelectrolyte coating layer 30, and drugs 40. At least one of the first polyelectrolyte coating layers 20 and at least one of the second polyelectrolyte coating layers 30 are formed on the gel matrix 10.

The ophthalmic lens 100 may be a hydrogel lens or a silicon hydrogel lens. When the ophthalmic lens 100 is a hydrogel lens, the gel matrix 10 is a hydrogel matrix. When the ophthalmic lens 100 is a silicon hydrogel lens, the gel matrix 10 is a silicon hydrogel matrix.

The gel matrix 10 and the first polyelectrolyte coating layer 20 have opposing electric charges <Please S&R>. The gel matrix 10 and the second polyelectrolyte coating layer 30 have same electrical charges.

When the gel matrix 10 includes ions with negative charges, the first polyelectrolyte coating layer 20 includes ions with positive charges, and the second polyelectrolyte coating layer 30 includes ions with negative charges. Acidity coefficient of the first polyelectrolyte coating layer 20 with positive charges (alkali compounds) pKa>7.4, acidity coefficient of the second polyelectrolyte coating layer 30 with negative charges (acidic compounds) pKa≤7.0.

When the gel matrix 10 includes ions with positive charges, the first polyelectrolyte coating layer 20 includes ions with negative charges, the second polyelectrolyte coating layer 30 includes ions with positive charges. Acidity coefficient of the first polyelectrolyte coating layer 20 with positive charges (alkali compounds) pKa≤7.0, acidity coefficient of the second polyelectrolyte coating layer 30 with negative charges (acidic compounds) pKa>7.4.

In at least one exemplary embodiment, the ophthalmic lens 100 includes a plurality of first polyelectrolyte coating layers 20 and a plurality of second polyelectrolyte coating layers 30.

One first polyelectrolyte coating layer 20 is formed on one surface of the gel matrix 10, the first polyelectrolyte coating layers 20 and the second polyelectrolyte coating layers 30 are set alternately.

The number of the first polyelectrolyte coating layers 20 in one surface of the gel matrix 10 is equal to number of the second polyelectrolyte coating layers 30 in one surface of the gel matrix 10. That is, one of the second polyelectrolyte coating layers 30 is formed on an outermost surface of the ophthalmic lens 100.

In detail, the number of the first polyelectrolyte coating layers 20 in one surface of the gel matrix 10 is great than or equal to 3. For example, the sum of the number of the first polyelectrolyte coating layers 20 in one surface of the gel matrix 10 and the number of the second polyelectrolyte coating layers 30 in one surface of the gel matrix 10 is greater than or equal to 6, to make the first polyelectrolyte coating layers 20 and the second polyelectrolyte coating layers 30 have a more stable structure.

In detail, in at least one exemplary embodiment, the number of the first polyelectrolyte coating layers 20 in one surface of the gel matrix 10 is 10.

In other exemplary embodiment, the ophthalmic lens 100 only includes a single first polyelectrolyte coating layer 20 and a single second polyelectrolyte coating layer 30. The first polyelectrolyte coating layer 20 is formed on one surface of the gel matrix 10. The second polyelectrolyte coating layer 30 is formed on the first polyelectrolyte coating layer 20. The second polyelectrolyte coating layer 30 faces away from the gel matrix 10.

The drugs 40 are formed or installed between the gel matrix 10 and the first polyelectrolyte coating layer 20, and between the first polyelectrolyte coating layer 20 and the second polyelectrolyte coating layer 30.

For acidic compounds, only when pH>pKa, the acidic compounds can exist in the form of ions. For alkali compounds, only when pH<pKa, the alkali compounds can exist in form of ions.

In at least one exemplary embodiment, the gel matrix 10 includes ions with negative charges, the first polyelectrolyte coating layer 20 includes ions with positive charges, and the second polyelectrolyte coating layer 30 includes ions with negative charges. So, in at least one exemplary embodiment, the pKa of the first polyelectrolyte coating layer 20 with positive charges is more than 7.4, the pKa of the second polyelectrolyte coating layer 30 with negative charges pKa is equal to or less than 7.0.

At normal physical conditions, pH of aqueous humor is 7.4. The first polyelectrolyte coating layer 20 and the second polyelectrolyte coating layer 30 interact electrostatically.

In at least one exemplary embodiment, because the pKa of the first polyelectrolyte coating layer 20 with positive charges is more than 7.4, the pH of aqueous humor is reduced from 7.4 to 7.0 when TOP rises. And then, when the pH of aqueous humor is 7.0, the pH of the aqueous humor is less than the pKa of the first polyelectrolyte coating layer 20 with positive charges. So when the pH of aqueous humor is 7.0, the first polyelectrolyte coating layer 20 (alkali compounds) can still exist in form of ions. That is to say, so when the pH of aqueous humor is 7.0, the first polyelectrolyte coating layer 20 still includes ions with positive charges.

When the pKa of the second polyelectrolyte coating layer 30 with negative charges pKa is equal to or less than 7.0, the pH of aqueous humor is reduced from 7.4 to 7.0 when IOP rises. And then, when the pH of aqueous humor is 7.0, the pH of aqueous humor is equal to the pKa of the first polyelectrolyte coating layer 20 with positive charges. Therefore, when the pH of aqueous humor is 7.0, the second polyelectrolyte coating layer 30 cannot exist in form of ions. So, when the pH of aqueous humor is 7.0, the result is low ionization degree of the second polyelectrolyte coating layer 30. At this moment, the electrostatic interaction between the first polyelectrolyte coating layer 20 and the second polyelectrolyte coating layer 30 is broken, and then the drugs 40 between the first polyelectrolyte coating layer 20 and the second polyelectrolyte coating layer 30 are released.

In at least one exemplary embodiment, the ions with negative charges in the gel matrix 10 may be —O⁻, —COO⁻, —SO₃⁻, —PO₄²⁻, —HPO₄⁻, and others.

In at least one exemplary embodiment, the ions with positive charges in the first polyelectrolyte coating layer 20 may be —NH₃⁺, and others.

In at least one exemplary embodiment, the ions with negative charges in the second polyelectrolyte coating layer 30 may be —O⁻, —COO⁻, —PO₄²⁻, —HPO₄⁻, and others.

In at least one exemplary embodiment, the ions with negative charges in the gel matrix 10 is —COO⁻.

In at least one exemplary embodiment, the ions with positive charges in the first polyelectrolyte coating layer 20 is Polyallylamine (PAH).

Molecular structural formula the PAH is:

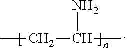

In at least one exemplary embodiment, the ions with negative charges in the second polyelectrolyte coating layer 30 is Polymethacrylic acid (PMAA).

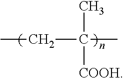

Molecular structural formula the PMAA is:

When the pH of aqueous humor is 7.4, the PAH and the PMAA interact by electrostatic interaction.

Principle of electrostatic interaction between the PAH and the PMAA is:

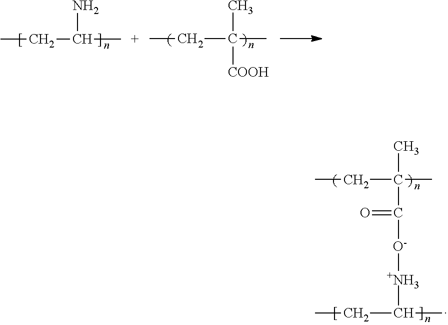

When the pH of aqueous humor is 7.0, the PAH coating layer (the first polyelectrolyte coating layer 20) still includes ions with positive charges, the PMAA coating layer (the second polyelectrolyte coating layer 30) has a low ionization degree, and the electrostatic interaction between the PAH coating layer (the first polyelectrolyte coating layer 20) and the PMAA coating layer (the second polyelectrolyte coating layer 30) is broken. The positive charges in the PAH coating layer (the first polyelectrolyte coating layer 20) then repel each other, and the positive charges in the PAH coating layer (the first polyelectrolyte coating layer 20) become intumescent, and then the drugs 40 between the first polyelectrolyte coating layer 20 and the second polyelectrolyte coating layer 30 are released.

Principle of cessation of electrostatic interaction between the PAH and the PMAA is:

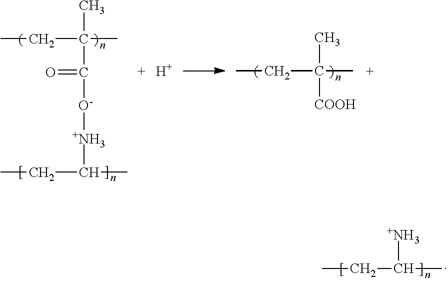

The drugs 40 are used to treat glaucoma.

In at least one exemplary embodiment, the drugs 40 may be beta-blocker, alpha-2 adrenergic agonist, carbonic anhydrase inhibitor, or prostaglandin analogue.

The following is an example of a method for manufacturing the ophthalmic lens 100. In the method, the number of the first polyelectrolyte coating layers 20 is n, and n=10.

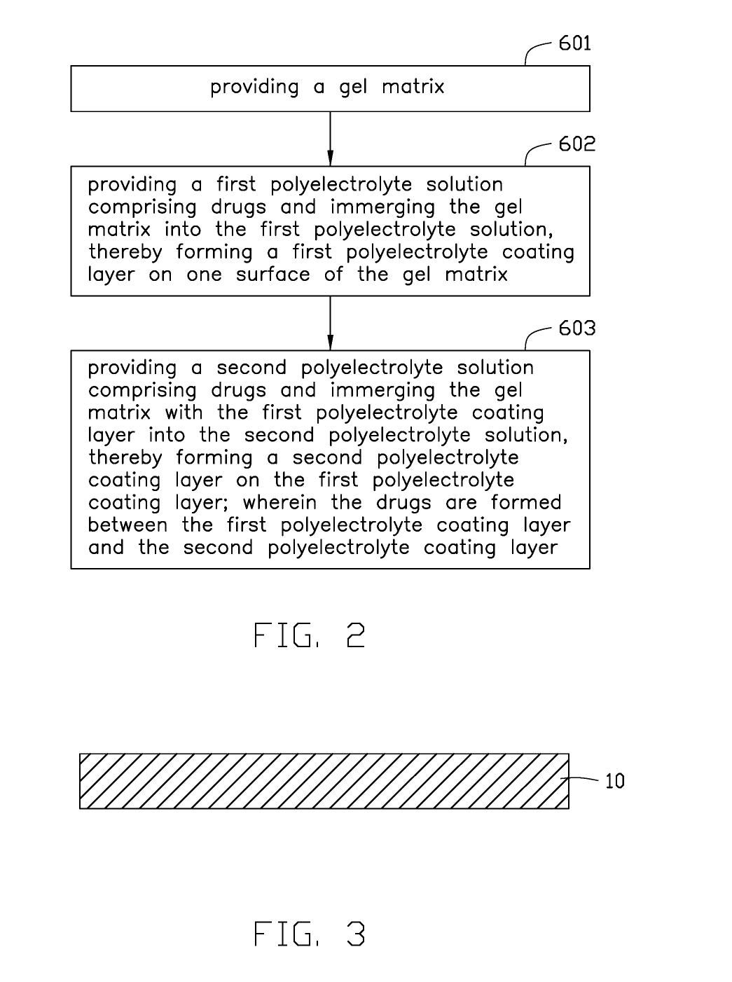

FIG. 2 illustrates a flowchart of a method for manufacturing an ophthalmic lens 100. The method is provided by way of example, as there are a variety of ways to carry out the method. The method described below may be carried out using the configurations illustrated in FIGS. 1-5, for example, and various elements of these figures are referenced in explaining example method. Each block shown in FIG. 2 represents one or more processes, methods, or subroutines, carried out in the exemplary method. Furthermore, the illustrated order of blocks is by example only and the order of the blocks can change. Additional blocks may be added or fewer blocks may be utilized, without departing from this disclosure. The exemplary method may begin at block 601.

At block 601, also illustrated by FIG. 3, a gel matrix 10 is provided.

The gel matrix 10 includes positive ions or negative ions.

In at least one exemplary embodiment, the positive ions or negative ions may be added into the gel matrix 10 by plasma treatment.

In at least one exemplary embodiment, the gel matrix 10 includes ions with negative charges. In at least one exemplary embodiment, the ions with negative charges in the gel matrix 10 may be —O⁻, —COO⁻, —SO₃⁻, —PO₄²⁻, —HPO₄⁻, and others.

The ophthalmic lens 100 may be a hydrogel lens or a silicon hydrogel lens. When the ophthalmic lens 100 is a hydrogel lens, the gel matrix 10 is a hydrogel matrix. When the ophthalmic lens 100 is a silicon hydrogel lens, the gel matrix 10 is a silicon hydrogel matrix.

Hydrogel precursors may be copolymerized to generate the hydrogel matrix under ultraviolet radiation. The hydrogel precursors include hydrated polymers, photoinitiators, and crosslinking agents. Silicon hydrogel precursors may be copolymerized to generate the silicon hydrogel matrix under ultraviolet radiation. The silicon hydrogel precursors include hydrated polymers, photoinitiators, and crosslinking agents.

When the gel matrix 10 is a hydrogel matrix, the hydrated polymers may be selected from methyl methacrylate (MMA) and hydroxyethyl methylacrylate (HEMA), or any combination thereof.

When the gel matrix 10 is a silicon hydrogel matrix, the hydrated polymers may be selected from one or more of methyl methacrylate (MMA), hydroxyethyl methylacrylate (HEMA) and polydimethylsiloxane (PDMS) and 3-methacrylic acid propyl tri-(tri-methoxysilane) (TRIS), or any combination thereof.

In at least one exemplary embodiment, the photoinitiators may include Darocur-1173, Darocur-2959, and Irgacure-1173, or any combination thereof.

In at least one exemplary embodiment, the cross-linking agent may include ethylene glycol dimethacrylate (EGDMA), trimethylolpropane trimethacrylate (TMPTMA), tri(ethylene glycol) dimethacrylate (TEGDMA), tri(ethylene glycol) divinyl ether (TEGDVE), and trimethylene glycol dimethacrylate, or any combination thereof.

The hydrogel precursors and the silicon hydrogel precursors also can include hydrophilic monomers.

When the gel matrix 10 is a hydrogel matrix, the hydrophilic monomers may include N-vinyl-2-pyrrolidone (NVP), glycidyl methacrylate (GMA), N, N-dimethylacrylamide (DMAA), or any combination thereof.

When the gel matrix 10 is a silicon hydrogel matrix, the hydrophilic monomers may include N-vinyl-2-pyrrolidone (NVP).

In at least one exemplary embodiment, the gel matrix 10 is a hydrogel matrix with —COO⁻ ions.

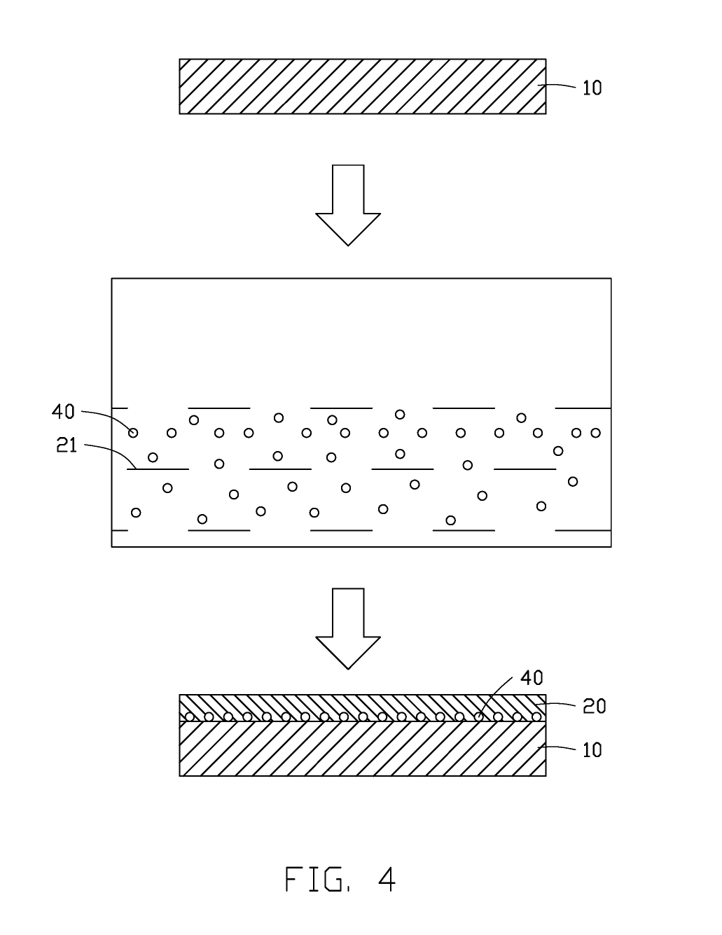

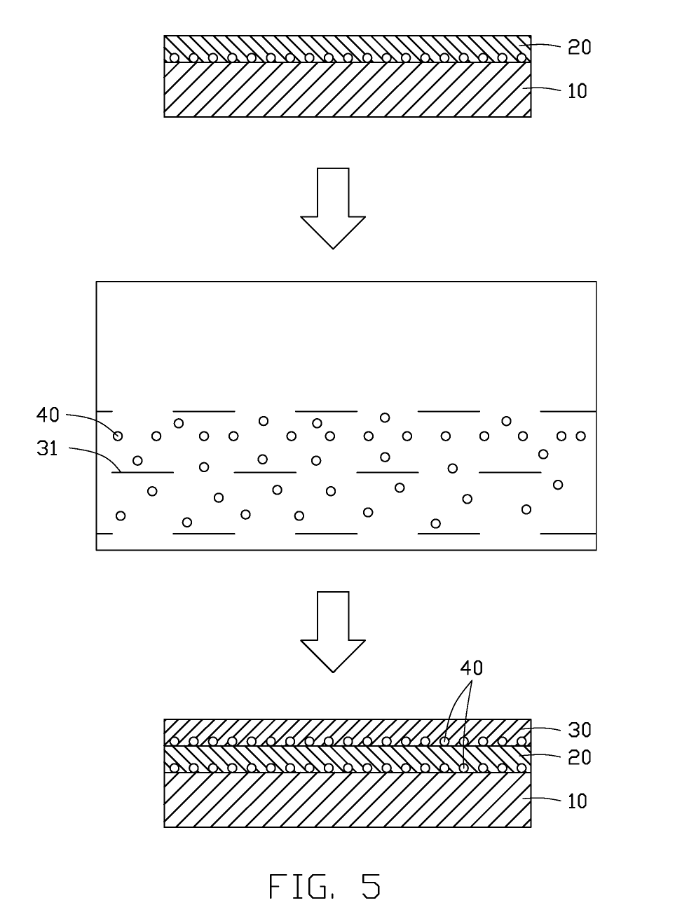

At block 602, also illustrated by FIGS. 4-5, a first polyelectrolyte solution 21 including drugs 40 and a second polyelectrolyte solution 31 including drugs 40 are provided. And then, the gel matrix 10 is immersed in the first polyelectrolyte solution 21 for a certain time to receive a first polyelectrolyte coating layer 20 on one surface of the gel matrix 10. And then, the gel matrix 10 with the first polyelectrolyte coating layer 20 is immersed in the second polyelectrolyte solution 31 for a certain time to receive a second polyelectrolyte coating layer 30 on the first polyelectrolyte coating layer 20.

PH of the first polyelectrolyte solution 21 and the second polyelectrolyte solution 31 are all 7.4.

The gel matrix 10 and polyelectrolyte of the first polyelectrolyte solution 21 have opposing electrical charges. The gel matrix 10 and polyelectrolyte of the second polyelectrolyte solution 31 have same electrical charges.

In at least one exemplary embodiment, the gel matrix 10 includes ions with negative charges.

In at least one exemplary embodiment, the ions with negative charges in the gel matrix 10 may be —O⁻, —COO⁻, —SO₃⁻, —PO₄²⁻, —HPO₄⁻, and others.

In at least one exemplary embodiment, polyelectrolyte of the first polyelectrolyte solution 21 includes ions with positive charges.

In at least one exemplary embodiment, the ions with positive charges in the first polyelectrolyte coating layer 20 may be —NH₃⁺, and others.

In at least one exemplary embodiment, the polyelectrolyte of the first polyelectrolyte solution 21 and the gel matrix 10 interact by electrostatic interaction, thereby forming a first polyelectrolyte coating layer 20.

Because molecules of the drugs 40 in the first polyelectrolyte solution 21 are microscopic molecules, some of the drugs 40 in the first polyelectrolyte solution 21 are formed between the first polyelectrolyte coating layer 20 and the gel matrix 10.

In other exemplary embodiment, the polyelectrolyte of the first polyelectrolyte solution 21 also may include ions with negative charges.

In at least one exemplary embodiment, polyelectrolyte of the second polyelectrolyte solution 31 includes ions with negative charges.

When the gel matrix 10 with the first polyelectrolyte coating layer 20 is immersed in the second polyelectrolyte solution 31, the polyelectrolyte of the second polyelectrolyte solution 31 and the first polyelectrolyte coating layer 20 interact by electrostatic interaction, thereby forming a second polyelectrolyte coating layer 30 on one surface of the first polyelectrolyte coating layer 20. The second polyelectrolyte coating layer 30 faces away from the gel matrix 10.

Because molecules of the drugs 40 in the second polyelectrolyte solution 31 are microscopic molecules, some of the drugs 40 in the second electrolyte solution 31 are formed between the first polyelectrolyte coating layer 20 and the second polyelectrolyte coating layer 30.

In at least one exemplary embodiment, the ions with negative charges in the second polyelectrolyte coating layer 30 may be —O⁻, —COO⁻, —PO₄²⁻, —HPO₄⁻, and others.

In other exemplary embodiment, the polyelectrolyte of the second polyelectrolyte solution 31 includes ions with positive charges.

In at least one exemplary embodiment, the pKa of the polyelectrolyte of the first polyelectrolyte solution 21 with positive charges is more than 7.4, and the pKa of the polyelectrolyte of the second polyelectrolyte solution 31 with negative charges pKa is equal to or less than 7.0.

The polyelectrolyte of the first polyelectrolyte solution 21 has a mass percentage of about 0.01% to about 1% of a total mass of the first polyelectrolyte solution 21. The drugs 40 in the first polyelectrolyte solution 21 have a mass percentage of about 0.015% to about 0.5% of a total mass of the first polyelectrolyte solution 21.

The polyelectrolyte of the second polyelectrolyte solution 31 has a mass percentage of about 0.01% to about 1% of a total mass of the second polyelectrolyte solution 31. The drugs 40 in the second polyelectrolyte solution 31 have a mass percentage of about 0.015% to about 0.5% of a total mass of the second polyelectrolyte solution 31.

In at least one exemplary embodiment, the polyelectrolyte of the first polyelectrolyte solution 21 is polyallylamine (PAH).

Molecular structural formula of the PAH is:

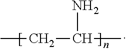

In at least one exemplary embodiment, the polyelectrolyte of the second polyelectrolyte solution 31 is polymethacrylic acid (PMAA).

Molecular structural formula of the PMAA is:

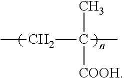

When the pH of aqueous humor is 7.4, the PAH and the PMAA interact electrostatically.

Principle of electrostatic interaction between the PAH and the PMAA is:

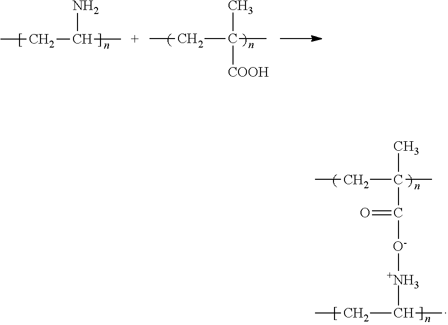

The drugs 40 are used to treat glaucoma.

The drugs 40 may be beta-blocker, alpha-2 adrenergic agonist, carbonic anhydrase inhibitor, or prostaglandin analogue.

In at least one exemplary embodiment, the drug 40 is brimonidine tartrate, the brimonidine tartrate is one of the alpha-2 adrenergic agonists, a new drug to treat glaucoma.

Further, after forming the first polyelectrolyte coating layer 20, the gel matrix 10 with the first polyelectrolyte coating layer 20 is washed by deionized water.

Further, after forming the second polyelectrolyte coating layer 30, the gel matrix 10 with the second polyelectrolyte coating layer 30 is also washed by deionized water.

The deionized water is used to wash out the redundant first polyelectrolyte coating layer 20 or the second polyelectrolyte coating layer 30 from the gel matrix 10.

At block 603, also illustrated by FIG. 1, the steps in block 602 are repeated, thereby forming a laminated construction about the first polyelectrolyte coating layer 20 and the second polyelectrolyte coating layer 30, thereby forming the ophthalmic lens 100.

Number of repetitions is determined according to actual needs.

In detail, the number of the first polyelectrolyte coating layers 20 in one surface of the gel matrix 10 is greater than or equal to 3. For example, the sum of the number of the first polyelectrolyte coating layers 20 in one surface of the gel matrix 10 and the number of the second polyelectrolyte coating layers 30 in one surface of the gel matrix 10 is greater than or equal to 6, to make the first polyelectrolyte coating layer 20 and the second polyelectrolyte coating layer 30 have a more stable structure.

In detail, in at least one exemplary embodiment, the number of the first polyelectrolyte coating layers 20 in one surface of the gel matrix 10 is 10.

The present disclosure will be described further by way of specific examples.

### Example 1

A gel matrix 10, a PAH solution (first polyelectrolyte solution 21), a PMAA solution (second polyelectrolyte solution 31), and deionized water are provided. The PAH solution includes brimonidine tartrate. The PMAA solution includes brimonidine tartrate. The pH of the PAH solution and the pH of the PMAA solution are all 7.4. The PAH has a mass percentage of about 1% of a total mass of the PAH solution. The brimonidine tartrate of the PAH solution has a mass percentage of about 0.02% of a total mass of the PAH solution. The PMAA has a mass percentage of about 1% of a total mass of the PMAA solution. The brimonidine tartrate of the PMAA solution has a mass percentage of about 0.02% of a total mass of the PMAA solution.

The gel matrix 10 is immersed in the PAH solution for 10 minutes, then the gel matrix 10 is taken out of the PAH solution and washed by deionized water, thereby forming a first polyelectrolyte coating layer 20 on one surface of the gel matrix 10.

Thereafter, the gel matrix 10 with the first polyelectrolyte coating layer 20 is immersed into the PMAA solution for 10 minutes, and then, the gel matrix 10 with the first polyelectrolyte coating layer 20 is taken out of the PMAA solution and washed by deionized water, thereby forming a second polyelectrolyte coating layer 30 on the first polyelectrolyte coating layer 20.

Thereafter, the gel matrix 10 with the first polyelectrolyte coating layer 20 and the second polyelectrolyte coating layer 30 is immersed in the PAH solution and the PMAA solution repeatedly, thereby forming a laminated construction about the first polyelectrolyte coating layer 20 and the second polyelectrolyte coating layer 30, thereby forming the ophthalmic lens 100.

In at least one exemplary embodiment, the number of the first polyelectrolyte coating layers 20 in one surface of the gel matrix 10 is 10.

With the above configuration, the first polyelectrolyte coating layer 20 is formed on the gel matrix 10, and the second polyelectrolyte coating layer 30 is formed on the first polyelectrolyte coating layer 20. The drugs 40 are formed between the first polyelectrolyte coating layer 20 and the second polyelectrolyte coating layer 30, and the first polyelectrolyte coating layer 20 and the second polyelectrolyte coating layer 30 interact electrostatically. So, when IOP rises, the pH of aqueous humor is reduced from 7.4 to 7.0. At this time, the electrostatic interaction between the first polyelectrolyte coating layer 20 and the second polyelectrolyte coating layer 30 is broken, and then the drugs 40 between the first polyelectrolyte coating layer 20 and the second polyelectrolyte coating layer 30 are released. Therefore, the ophthalmic lens 100 in the present disclosure can timely detect TOP level to achieve a real-time drug release.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. An ophthalmic lens, comprising:
a gel matrix;
at least one first polyelectrolyte coating layer, wherein the first polyelectrolyte coating layer is formed on the gel matrix;
at least one second polyelectrolyte coating layer, wherein the second polyelectrolyte coating layer is formed on the first polyelectrolyte coating layer; and
drugs, wherein the drugs are formed between the first polyelectrolyte coating layer and the second polyelectrolyte coating layer;
wherein the first polyelectrolyte coating layer and the second polyelectrolyte coating layer interact electrostatically; wherein the electrostatic interaction between the first polyelectrolyte coating layer and the second polyelectrolyte coating layer is broken when TOP rises, the drugs are released.

2. The ophthalmic lens of claim 1, wherein the gel matrix and the first polyelectrolyte coating layer have opposing electrical charges, the gel matrix and the second polyelectrolyte coating layer have same electrical charges.

3. The ophthalmic lens of claim 2, wherein the gel matrix comprises ions with negative charges, the first polyelectrolyte coating layer comprises ions with positive charges, the second polyelectrolyte coating layer comprises ions with negative charges.

4. The ophthalmic lens of claim 3, wherein the ions with positive charges in the first polyelectrolyte coating layer is Polyallylamine (PAH) coating layer.

5. The ophthalmic lens of claim 3, wherein the ions with negative charges in the second polyelectrolyte coating layer is polymethacrylic acid (PMAA) coating layer.

6. The ophthalmic lens of claim 2, wherein the gel matrix comprises ions with positive charges, the first polyelectrolyte coating layer comprises ions with negative charges, and the second polyelectrolyte coating layer comprises ions with positive charges.

7. The ophthalmic lens of claim 1, wherein the ophthalmic lens only comprises a single first polyelectrolyte coating layer and a single second polyelectrolyte coating layer.

8. The ophthalmic lens of claim 1, wherein the ophthalmic lens comprises a plurality of first polyelectrolyte coating layers and a plurality of second polyelectrolyte coating layers, the first polyelectrolyte coating layers and the second polyelectrolyte coating layers are set alternately on the gel matrix.

9. The ophthalmic lens of claim 1, wherein a number of the first polyelectrolyte coating layers is equal to a number of the second polyelectrolyte coating layers.

10. The ophthalmic lens of claim 7, wherein a number of the first polyelectrolyte coating layers in one surface of the gel matrix is greater than or equal to 3.

11. The ophthalmic lens of claim 1, wherein the drugs are beta-blocker, alpha-2 adrenergic agonist, carbonic anhydrase inhibitor, or prostaglandin analogue.

12. A method for manufacturing an ophthalmic lens, comprising:
providing a gel matrix;
providing a first polyelectrolyte solution comprising drugs and immersing the gel matrix into the first polyelectrolyte solution, thereby forming a first polyelectrolyte coating layer on one surface of the gel matrix; and
providing a second polyelectrolyte solution comprising drugs and immersing the gel matrix with the first polyelectrolyte coating layer into the second polyelectrolyte solution, thereby forming a second polyelectrolyte coating layer on the first polyelectrolyte coating layer;
wherein the drugs are formed or installed between the first polyelectrolyte coating layer and the second polyelectrolyte coating layer.

13. The ophthalmic lens of claim 12, wherein pH of the first polyelectrolyte solution and the second polyelectrolyte solution are 7.4.

14. The ophthalmic lens of claim 12, wherein the gel matrix and polyelectrolyte of the first polyelectrolyte solution have opposing electrical charges, the gel matrix and polyelectrolyte of the second polyelectrolyte solution have same electrical charges.

15. The ophthalmic lens of claim 12, wherein polyelectrolyte of the first polyelectrolyte solution has a mass percentage of about 0.01% to about 1% of a total mass of the first polyelectrolyte solution, the drugs in the first polyelectrolyte solution has a mass percentage of about 0.015% to about 0.5% of a total mass of the first polyelectrolyte solution.

16. The ophthalmic lens of claim 12, wherein polyelectrolyte of the second polyelectrolyte solution has a mass percentage of about 0.01% to about 1% of a total mass of the second polyelectrolyte solution, the drugs in the second polyelectrolyte solution has a mass percentage of about 0.015% to about 0.5% of a total mass of the second polyelectrolyte solution.

17. The ophthalmic lens of claim 12, wherein the drugs are beta-blocker, alpha-2 adrenergic agonist, carbonic anhydrase inhibitor, or prostaglandin analogue.

18. The ophthalmic lens of claim 12, after forming the first polyelectrolyte coating layer, further comprising:
washing the gel matrix with the first polyelectrolyte coating layer by deionized water.

19. The ophthalmic lens of claim 12, after forming the second polyelectrolyte coating layer, further comprising:
washing the gel matrix with the second polyelectrolyte coating layer by deionized water.

20. The ophthalmic lens of claim 12, after forming the second polyelectrolyte coating layer, further comprising:
immersing the gel matrix with the first polyelectrolyte coating layer and the second polyelectrolyte coating layer into the first polyelectrolyte solution and the second polyelectrolyte solution repeatedly, thereby forming a laminated construction about the first polyelectrolyte coating layers and the second polyelectrolyte coating layers, thereby forming the ophthalmic lens.
